(19) United States
(12) Patent Application Publication
(10) Pub. No.: US 2024/0392328 A1
(43) Pub. Date: Nov. 28, 2024

(54) COMPREHENSIVE CARBON DIOXIDE AND CARBON MONOXIDE SEQUESTRATION PROCESS AND SYSTEM

(71) Applicant: Jupeng Bio (HK) Limited, Hong Kong (CN)

(72) Inventors: Ryan Senaratne, Fayetteville, AR (US); Ke Li, League City, TX (US)

(73) Assignee: Jupeng Bio (HK) Limited, Hong Kong (CN)

(21) Appl. No.: 18/671,169

(22) Filed: May 22, 2024

Related U.S. Application Data
(60) Provisional application No. 63/468,601, filed on May 24, 2023.

(51) Int. Cl. C12P 7/16; C12M 1/00; C12M 1/107; C12N 1/20; C12P 7/06; C12R 1/02; C12R 1/145

(52) U.S. Cl. CPC C12P 7/16; C12M 21/04; C12M 43/08; C12N 1/20; C12P 7/06; C12R 2001/02; C12R 2001/145

(57) ABSTRACT

System and process are provided for producing and obtaining valuable products that are derived from microbial fermentation of C1-containing gaseous substrate process. More specifically, the disclosure provides schemes for the integration of a fermentation process with electrolysis process and C1-containing gaseous substrate generating process while achieving reduced carbon dioxide emission. The amount of carbon dioxide subject to the electrolysis process is based on the balance of raw material consumption of the microbial fermentation process.

This application claims the benefit of U.S. Provisional Application No. 63/468,601, filed May 24, 2023, which is incorporated in its entirety herein by reference.

A process is provided for biologically sequestrating carbon dioxide and carbon monoxide to produce useful products, materials, intermediates, and the like such as oxygenated hydrocarbonaceous compound and single cell protein. More specifically, the process includes producing organic acids, organic acid salts, alcohols, and protein supplement with reduced carbon dioxide emission.

### BACKGROUND

Carbon monoxide (CO) and carbon dioxide (CO₂) emissions are two of the major drivers of climate change and global warming. Microbial fermentation can reduce carbon monoxide emission by utilizing microorganisms, through their metabolic pathways, to convert carbon monoxide (CO) and hydrogen (H₂) into useful oxygenated hydrocarbonaceous compounds, such as ethanol, butanol, acetate, butyrate, 2,3-butanediol, and other desired products.

Syngas, comprising carbon monoxide (CO), hydrogen (H₂) and carbon dioxide (CO₂), is formed by gasification of carbonaceous materials. It is believed that microbial fermentation of syngas to oxygenated hydrocarbonaceous compounds can decrease its carbon footprint on the environment. Carbon dioxide contained in the syngas, generated during the bioconversion of carbon monoxide and hydrogen, and/or collected from other industrial processes may be converted into organic acid in subsequent microbial fermentation steps. However, it is still difficult to effectively utilize carbon dioxide in biological processes because of its highly oxidized state.

In view of the large amount of carbon monoxide and carbon dioxide generated, there is a need for a comprehensive biological carbon sequestration process and system that can utilize both carbon monoxide and carbon dioxide to produce useful products while having reduced or negative carbon dioxide emission.

### SUMMARY

In accordance with the present disclosure, system and process are provided for effectively producing useful oxygenated hydrocarbonaceous compounds from fermenting carbon monoxide and carbon dioxide from syngas and/or industrial off-gas with reduced carbon dioxide emission.

A fermentation process includes providing a feedstock to a gasifier to generate a CO-containing syngas. The CO-containing syngas is passed to a bioconversion system with at least one CO conversion bioreactor and one CO₂ conversion bioreactor. Alcohol is recovered from the bioconversion system. The process then includes passing a CO₂-containing stream from the bioconversion system to a CO₂ electrolyzer to produce a first O₂ stream and a CO stream. The first O₂ stream is then provided to the gasifier and the CO stream is passed to the bioconversion system.

A fermentation system includes a gasifier gasifying a feedstock to generate a CO-containing syngas and a bioconversion system with at least one CO conversion bioreactor and one CO₂ conversion bioreactor receiving the CO-containing syngas and producing an alcohol and a CO₂-containing stream. The system further includes a CO₂ electrolyzer electrolyzing the CO₂-containing stream to produce a first O₂ stream and a CO stream. A pipeline is placed to recycle the CO stream to the bioconversion system and another pipeline is placed to recycle the first O₂ stream to the gasifier.

A fermentation process includes providing a feedstock to a gasifier to generate a CO-containing syngas with 20% or more CO₂ and 25% or less CO. The CO-containing syngas is separated into a CO₂ stream and a CO-concentrated fermentable syngas. The CO-concentrated fermentable syngas is then passed to a CO conversion bioreactor to produce a cell free alcohol-containing permeate and a CO₂-containing vent gas. Alcohol is recovered from the cell free alcohol-containing permeate. The process then includes passing at least a portion of the CO₂ stream and the CO₂-containing vent gas to a CO₂ conversion bioreactor to produce a cell free organic acid-containing permeate and a CO₂-containing exhaust gas. The cell free organic acid-containing permeate is then delivered to the CO conversion bioreactor.

A fermentation process is provided. The process includes providing a CO-containing gaseous substrate to a CO conversion bioreactor with a CO converting anaerobic bacteria. The CO conversion bioreactor then produces a cell free alcohol-containing permeate and a vent gas. Alcohol is recovered from the cell free alcohol-containing permeate. At least a portion of the vent gas is sent to a CO₂ electrolyzer to produce a CO stream. The CO stream is then provided back to the CO conversion bioreactor. The process further includes fermenting at least a portion of the vent gas in a CO₂ conversion bioreactor with a CO₂ converting anaerobic bacteria to produce a cell free organic acid permeate and an exhaust gas and recycling at least a portion of the cell free organic acid-containing permeate to the CO conversion bioreactor.

A fermentation system includes a CO conversion bioreactor fermenting an organic acid and a CO-containing gaseous substrate with a CO converting anaerobic bacteria to produce a cell free alcohol-containing permeate and a vent gas, and a CO₂ conversion bioreactor fermenting at least a portion of the vent gas with a CO₂ converting anaerobic bacteria to produce a cell free organic acid-containing permeate and an exhaust gas. A pipeline is placed to recycle at least a portion of the cell free organic acid-containing permeate to the CO conversion bioreactor. The system further includes a distillation column to recover alcohol from the cell free alcohol-containing permeate. A CO₂ electrolyzer is then used to electrolyze at least a portion of the vent gas to generate a CO stream and a pipeline is placed to recycle the CO stream to the CO conversion bioreactor.

A fermentation process includes providing a biogenic material to an incinerator to produce a CO₂-containing gaseous substrate. At least a portion of the CO₂-containing gaseous substrate is fermented in a CO₂ conversion bioreactor with a CO₂ converting anaerobic bacteria to produce a cell free organic acid-containing permeate and an exhaust gas. The process further includes delivering a CO₂ stream to a CO₂ electrolyzer to produce a first O₂ stream and a CO stream. The first O₂ stream is passed back to the incineration. The CO stream and at least a portion of the cell free organic acid-containing permeate is then fermented with a CO converting anaerobic bacteria in a CO conversion bioreactor to produce a cell free alcohol-containing permeate and a vent gas CO₂ stream. At least a portion of the vent gas CO₂ stream is delivered back to the CO₂ conversion bioreactor.

A fermentation system includes an incinerator to incinerate a biogenic material to produce a CO₂-containing incineration flue gas and a CO₂ conversion bioreactor fermenting at least a portion of the CO₂-containing incineration flue gas to produce a cell free organic acid-containing permeate and an exhaust gas. A CO₂ electrolyzer is configured to receive a CO₂ stream to produce a first O₂ stream and a CO stream. The first O₂ stream is recycled back to the incinerator through a pipeline. The fermentation system further includes a CO conversion bioreactor fermenting the CO stream and the cell free organic acid-containing permeate with a CO converting anaerobic bacteria to produce a cell free alcohol-containing permeate and a vent gas CO₂ stream. A pipeline is configured to recycle at least a portion of the vent gas CO₂ stream to the CO₂ conversion bioreactor.

### BRIEF DESCRIPTION OF FIGURES

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only typical embodiments of this disclosure and are therefore not to be considered limiting of its scope, for the disclosure may admit to other equally effective embodiments.

FIG. 1 illustrates a schematic of a system for producing an oxygenated hydrocarbonaceous compound with reduced carbon dioxide emissions.

FIG. 2 illustrates a schematic of a system for producing an oxygenated hydrocarbonaceous compound with reduced or negative carbon dioxide emissions.

FIG. 3 illustrates a schematic of a system for generating power and producing an oxygenated hydrocarbonaceous compound with reduced or negative carbon dioxide emissions.

FIG. 4 illustrates a schematic of a system for generating power and producing an oxygenated hydrocarbonaceous compound and a single cell protein with reduced or negative carbon dioxide emissions.

FIG. 5 illustrates a schematic of a system for sequestrating CO₂ with reduced or negative carbon dioxide emissions.

### DETAILED DESCRIPTION

The following description is not to be taken in a limiting sense, but is made merely for the purpose of describing the general principles of exemplary embodiments. The scope of the disclosure should be determined with reference to the claims.

The term “about” modifying any amount refers to the variation in that amount encountered in real world conditions, e.g. in the lab, pilot plant, or production facility. For example, an amount of an ingredient or measurement employed in a mixture or quantity when modified by “about” includes the variation and degree of care typically employed in measuring in an experimental condition in production plant or lab. For example, the amount of a component of a product when modified by “about” includes the variation between batches in multiple experiments in the plant or lab and the variation inherent in the analytical method. Whether or not modified by “about” the amounts include equivalents to those amounts. Any quantity stated herein and modified by “about” can also be employed in the present disclosure as the amount not modified by “about”.

The use of the terms “a”, “an”, “the” and similar referents in the context of this disclosure are to be construed to cover both the singular and the plural, unless otherwise indicated or clearly contradicted by context.

Unless otherwise indicated, the terms “comprising”, “including”, “having”, “containing”, or “characterized by” are inclusive and does not exclude any additional, unrecited elements or method steps (i.e. meaning “including, but not limited to”). The use of any and all examples or exemplary language (e.g., “such as”, “for example”, “for instance”) provided herein is intended merely to illuminate the disclosure and does not impose a limitation on the scope of the disclosure unless otherwise claimed.

Fermentation is a metabolic process used by bacteria to generate energy for cell growth. Certain anaerobic bacteria are capable of fermenting C1-containing gaseous substrate, such as fermentable syngas, carbon monoxide (CO) containing gaseous substrate, or carbon dioxide (CO₂) containing gaseous substrate, to sustain their growth and produce oxygenated hydrocarbonaceous compounds. The terms “fermentation”, “fermentation process”, “microbial fermentation process” and the like are intended to encompass both the growth phase and the product biosynthesis phase of the process. During an anaerobic bacterial fermentation process, large amounts of microbial biomass are obtained, which may be purged out and processed to useful products, such as nutrient supplements.

Anaerobic bacteria are bacteria that do not require oxygen for growth. An anaerobic bacteria may react negatively or even die if oxygen is present above certain threshold. Acetogenic bacteria are microorganisms that are capable of producing acetate under anaerobic respiration or fermentation by utilizing the Wood-Ljungdahl pathway as their main mechanism for energy conservation. Other useful oxygenated hydrocarbon compounds, such as formic acid, propionic acid, butyric acid, heptanoic acid, decanoic acid, ethanol, butanol, 2-butanol, and 2,3-butanediol, may also be produced by the acetogenic bacteria. Examples of the acetogenic bacteria suitable for converting C1-containing gaseous substrate to useful oxygenated hydrocarbonaceous compounds include those of the genus Clostridium, such as strains of Clostridium ljungdahlii, including those described in WO 2000/68407, EP 117309, U.S. Pat. Nos. 5,173,429, 5,593,886 and 6,368,819, WO 1998/00558 and WO 2002/08438, strains of Clostridium autoethanogenum (DSM 10061 and DSM 19630 of DSMZ, Germany) including those described in WO 2007/117157 and WO 2009/151342 and Clostridium ragsdalei (P11, ATCC BAA-622) and Alkalibaculum bacchi (CP11, ATCC BAA-1772) including those described respectively in U.S. Pat. No. 7,704,723 and “Biofuels and Bioproducts from Biomass-Generated Synthesis Gas”, Hasan Atiyeh, presented in Oklahoma EPSCOR Annual State Conference, Apr. 29, 2010 and Clostridium carboxidivorans (ATCC PTA-7827) described in U.S. Patent Application No. 2007/0276447. Other suitable microorganisms include those of the genus Moorella, including Moorella sp. HUC22-1, and those of the genus Carboxydothermus. Each of these references is incorporated herein by reference. Mixed cultures of two or more microorganisms may be used.

Additional examples of useful acetogenic bacteria include Acetogenium kivui, Acetoanaerobium noterae, Acetobacterium woodii, Alkalibaculum bacchi CP11 (ATCC BAA-1772), Blautia producta, Butyribacterium methylotrophicum, Caldanaerobacter subterraneous, Caldanaerobacter subterraneous pacificus, Carboxydothermus hydrogenoformans, Clostridium aceticum, Clostridium acetobutylicum, Clostridium acetobutylicum P262, Clostridium autoethanogenum (DSM 19630 of DSMZ Germany), Clostridium autoethanogenum (DSM 10061 of DSMZ Germany), Clostridium autoethanogenum (DSM 23693 of DSMZ Germany), Clostridium autoethanogenum (DSM 24138 of DSMZ Germany), Clostridium carboxidivorans P7 (ATCC PTA-7827), Clostridium coskatii (ATCC PTA-10522), Clostridium drakei, Clostridium ljungdahlii PETC (ATCC 49587), Clostridium ljungdahlii ERI2 (ATCC 55380), Clostridium ljungdahlii C-01 (ATCC 55988), Clostridium ljungdahlii O-52 (ATCC 55889), Clostridium magnum, Clostridium pasteurianum (DSM 525 of DSMZ Germany), Clostridium ragsdalei P11 (ATCC BAA-622), Clostridium scatologenes, Clostridium thermoaceticum, Clostridium ultunense, Desulfotomaculum kuznetsovii, Eubacterium limosum, Geobacter sulfurreducens, Methanosarcina acetivorans, Methanosarcina barkeri, Moorella thermoacetica, Moorella thermoautotrophica, Oxobacter pfennigii, Peptostreptococcus productus, Ruminococcus productus, Thermoanaerobacter kivui, Clostridium Stick-landii, and mixtures thereof.

The fermentation of the gaseous substrate with the acetogenic bacteria takes place in a bioreactor or fermentation vessel. Bioreactor/fermentation vessel includes one or more vessels and/or towers and/or piping arrangements, which includes a batch reactor, semi-batch reactor, continuous reactor, continuous stirred tank reactor (CSTR), bubble column reactor, external circulation loop reactor, internal circulation loop reactor, immobilized cell reactor (ICR), trickle bed reactor (TBR), moving bed biofilm reactor (MBBR), gas lift reactor, membrane reactor such as hollow fiber membrane bioreactor (HFMBR), static mixer, gas lift fermentor, or other vessel or other device suitable for gas-liquid contact.

A culture medium suitable for anaerobic bacterial growth and fermenting gaseous substrate into one or more oxygenated hydrocarbonaceous compounds can be added to the bioreactor to support the fermentation of the gaseous substrate by the acetogenic bacteria. Some examples of medium compositions are described in U.S. Ser. Nos. 16/530,502 and 16/530,481, filed Aug. 2, 2019, and in U.S. Pat. No. 7,285,402, filed Jul. 23, 2001, all of which are incorporated herein by reference. The medium may be sterilized to remove undesirable microorganisms and the bioreactor is inoculated with the desired microorganisms. Sterilization may not always be required.

The term “CO converting anaerobic bacteria” refers to the acetogenic bacteria that can convert carbon monoxide into useful oxygenated hydrocarbonaceous compounds, such as alcohols. CO converting anaerobic bacteria may not be able to effectively utilize CO₂ through its metabolic pathways and CO₂ is usually a byproduct of the CO bioconversion process. In this aspect, suitable CO-containing gaseous substrate contains at least about 5 mole % CO, in one aspect, at least about 10 mole %, in one aspect, at least about 20 mole %, in one aspect, at least about 25 mole %, in one aspect, at least about 30 mole %, in one aspect, about 10 to about 100 mole %, in another aspect, about 25 to about 100 mole % CO, in another aspect, about 30 to about 90 mole % CO, in another aspect, about 40 to about 80 mole % CO, and in another aspect, about 50 to about 70 mole % CO. In this aspect, the CO-containing gaseous substrate may have about 40 mole % or less CO₂, in one aspect, the CO-containing gaseous substrate may have about 30 mole % or less CO₂, in one aspect, the CO-containing gaseous substrate may have about 20 mole % or less CO₂, in another aspect, the CO-containing gaseous substrate may have about 10 mole % or less CO₂, in another aspect, the CO-containing gaseous substrate may have about 1 mole % or less CO₂, in still another aspect, the CO-containing gaseous substrate may comprise no or substantially no CO₂.

Depending on the composition of the CO-containing gaseous substrate, the CO-containing gaseous substrate may be directly provided to a CO bioconversion process or may be further modified or blended to include an appropriate H₂ to CO molar ratio. In one aspect, the CO-containing gaseous substrate provided to the bioreactor has an H₂ to CO molar ratio of about 0.1 or more, in another aspect, about 0.2 or more, in another aspect, about 0.3 or more, in another aspect, about 0.5 or more, in another aspect, about 0.8 or more, and in another aspect, about 1 or more.

The CO converting anaerobic bacteria can also convert organic acids into useful oxygenated hydrocarbonaceous compounds. In one aspect, both organic acids and CO-containing gaseous substrate are provided to CO bioconversion process and converted into oxygenated hydrocarbonaceous compounds by the CO converting anaerobic bacteria. For example, the CO converting anaerobic bacteria may convert acetic acid (CH₃COOH) and carbon monoxide (CO) into ethanol (C₂H₅OH) through the following stoichiometric equations:

$$6 CO + 3 {H}_{2} O \to {C}_{2} {H}_{5} OH + 4 {CO}_{2}$$

$$11 CO + {H}_{2} + 5 {H}_{2} O \to 2 {C}_{2} {H}_{5} OH + 7 {CO}_{2}$$

$$2 {CH}_{3} COOH + 3 CO + {H}_{2} O + {H}_{2} \to 2 {C}_{2} {H}_{5} OH + 3 {CO}_{2}$$

$${CH}_{3} COOH + CO + {H}_{2} \to {C}_{2} {H}_{5} OH + {CO}_{2}$$

$${CH}_{3} COOH + 2 CO + {H}_{2} O \to {C}_{2} {H}_{5} OH + 2 {CO}_{2}$$

Concentrations of various medium components for use in the CO bioconversion fermentation process are as follows:

| | Concentration | Feed Rate |
|---|---|---|
| Element | mg/L | μg/gram cells/min |
| NH₄⁺ | 164-6560 | 41-1640 |
| Fe | 1.7-68 | 0.425-17 |
| Ni | 0.07-2.81 | 0.017-0.702 |
| Co | 0.037-1.49 | 0.009-0.373 |
| Se | 0.027-1.1 | 0.006-0.274 |
| Zn | 0.116-4.64 | 0.198-5.95 |
| W | 0.8-32.1 | 0.26-8.03 |
| K | 39-1573 | 9.83-393.25 |
| Mg | 1.4-57.3 | 0.35-14.32 |
| S | 15-625 | 3.9-156.2 |
| P | 15-601 | 3.76-150.43 |
| d-biotin | 0.016-0.64 | 0.004-0.16 |
| thiamine HCl | 0.04-1.6 | 0.01-0.4 |
| calcium-D-pantothenate | 0.02-0.81 | 0.005-0.202 |

The CO bioconversion process is maintained in a pH range of about 4 to 6, in another aspect, 4 to 5, in another aspect, 4.2 to 4.8, in another aspect, 5 to 6, in another aspect, 5 to 5.5, and still in another aspect, 4.5 to 5.3. The medium includes no or substantially no yeast extract nor carbohydrates and carbon from the C1-containing gaseous substrate is the primary carbon source for the CO converting anaerobic bacteria.

Examples of useful CO converting anaerobic bacteria include Blautia producta, Butyribacterium methylotrophicum, Caldanaerobacter subterraneous, Caldanaerobacter subterraneous pacificus, Carboxydothermus hydrogenoformans, Clostridium aceticum, Clostridium acetobutylicum, Clostridium acetobutylicum P262, Clostridium autoethanogenum (DSM 19630 of DSMZ Germany), Clostridium autoethanogenum (DSM 10061 of DSMZ Germany), Clostridium autoethanogenum (DSM 23693 of DSMZ Germany), Clostridium autoethanogenum (DSM 24138 of DSMZ Germany), Clostridium carboxidivorans P7 (ATCC PTA-7827), Clostridium coskatii (ATCC PTA-10522), Clostridium drakei, Clostridium ljungdahlii PETC (ATCC 49587), Clostridium ljungdahlii ERI2 (ATCC 55380), Clostridium ljungdahlii C-01 (ATCC 55988), Clostridium ljungdahlii O-52 (ATCC 55889), Clostridium magnum, Clostridium pasteurianum (DSM 525 of DSMZ Germany), Clostridium ragsdalei P11 (ATCC BAA-622), Clostridium scatologenes, Clostridium thermoaceticum, Clostridium ultunense, Desulfotomaculum kuznetsovii, Eubacterium limosum, Geobacter sulfurreducens, Methanosarcina acetivorans, Methanosarcina barkeri, Oxobacter pfennigii, Peptostreptococcus productus, Thermoanaerobacter kivui, Clostridium Stick-landii, and mixtures thereof.

The term “CO₂ converting anaerobic bacteria” refers to the acetogenic bacteria that can utilize and convert carbon dioxide. CO₂ bioconversion process with CO₂ converting anaerobic bacteria consumes CO₂ and produces useful products, such as organic acids. In this aspect, the CO₂ bioconversion process is effective for producing about 25 to 100% conversion of CO₂, in another aspect, about 50% to 100% conversion of CO₂, in another aspect, about 75% to 100% conversion of CO₂, in another aspect, about 85% to 100% conversion of CO₂, in another aspect, about 90% to 100% conversion of CO₂, and in still another aspect, about 95% to 100% conversion of CO₂. In one aspect, suitable CO₂-containing gaseous substrate contains at least about 10 mole % CO₂, in one aspect, at least about 20 mole %, in one aspect, at least about 30 mole %, in one aspect, at least about 40 mole %, in one aspect, about 10 to about 70 mole %, in another aspect, about 20 to about 70 mole % CO₂, in another aspect, about 30 to about 70 mole % CO₂, in another aspect, about 40 to about 70 mole % CO₂, in another aspect, about 10 to about 50 mole % CO₂, in another aspect, about 20 to about 40 mole % CO₂, and in still another aspect, about 30 to 50 mole % CO₂. In this aspect, the CO₂-containing gaseous substrate contains about 50 mole % or less CO, in one aspect, the CO₂-containing gaseous substrate contains about 40 mole % or less CO, in one aspect, the CO₂-containing gaseous substrate contains about 30 mole % or less CO, in one aspect, the CO₂-containing gaseous substrate contains about 20 mole % or less CO, in one aspect, the CO₂-containing gaseous substrate contains about 10 mole % or less CO, in one aspect, the CO₂-containing gaseous substrate contains about 5 mole % or less CO, in one aspect, the CO₂-containing gaseous substrate contains about 1 mole % or less CO, in another aspect, the CO₂-containing gaseous substrate contains no or substantially no CO.

Depending on the composition of the CO₂-containing gaseous substrate, the CO₂-containing gaseous substrate may be directly provided to a fermentation process or may be further modified or blended to include an appropriate H₂ to CO₂ molar ratio. For example, a stream comprising a high concentration of CO₂, such as the exhaust from an industrial process, can be combined with a stream comprising high concentrations of H₂, such as the off gas from a coke oven. In one aspect, the gaseous substrate provided to the bioreactor has an H₂ to CO₂ molar ratio of about 4:1 to about 1:2, in another aspect, about 4:1 to about 1:1, in another aspect, about 4:1 to about 2:1, and in still another aspect, about 3.5:1 to about 1.5:1.

In one aspect, the CO₂ converting anaerobic bacteria converts CO₂ into acetic acid (CH₃COOH). In this aspect, the conversion is shown by the following stoichiometric equation:

$$2 {CO}_{2} + 4 {H}_{2} \to {CH}_{3} COOH + 2 {H}_{2} O$$

Concentrations of various medium components for use in the CO₂ bioconversion fermentation process are as follows:

| | Concentration | Feed Rate |
|---|---|---|
| Element | mg/L | μg/gram cells/min |
| NH₄⁺ | 82-3280 | 20.5-820 |
| Fe | 0.85-34 | 0.28-8.5 |
| Ni | 0.07-2.81 | 0.023-0.702 |
| Co | 0.037-1.49 | 0.012-0.373 |
| Se | 0.027-1.1 | 0.009-0.274 |
| Zn | 0.59-23.8 | 0.198-5.95 |
| Mo | 0.003-0.397 | 0.003-0.1 |
| chelator | 2.5-100 | 0.83-25 |
| W | 0.8-32.1 | 0.26-8.03 |
| K | 98-3933 | 32.77-983.35 |
| Mg | 0.71-28.69 | 0.23-7.18 |
| Na | 875-35000 | 290-8750 |
| S | 15-625 | 2.08-62.5 |
| P | 20-805 | 6.7-201.3 |
| d-biotin | 0.016-0.64 | 0.005-0.16 |
| thiamine HCl | 0.04-1.6 | 0.01-0.4 |
| calcium-D-pantothenate | 0.02-0.81 | 0.006-0.202 |

The CO₂ bioconversion process is maintained in a pH range of about 5 to 8, in another aspect, 5 to 6.2, in another aspect, 5.5 to 6.5, in another aspect, 6 to 7.5, in another aspect, 5.8 to 7.8, and still in another aspect, 6.5 to 8. The medium includes no or substantially no yeast extract nor carbohydrates and carbon from the C1-containing gaseous substrate is the primary carbon source for the CO₂ converting anaerobic bacteria.

Suitable CO₂ converting anaerobic bacteria may include a sodium pump which may also be described as sodium-translocating ATPases (for membrane bioenergetics). Sodium translocating ATPase are described in Muller, “Energy Conservation in Acetogenic Bacteria,” Appl. Environ. Microbiol. November 2003, vol. 69, no. 11, pp. 6345-6353, which is incorporated herein by reference. Acetogenic bacteria that include a sodium-translocating ATPase require about 500 ppm NaCl in their growth medium for growth. To determine if an acetogenic bacteria includes a sodium-translocating ATPase, the acetogen is inoculated into serum bottles containing about 30 to about 50 ml of growth medium with about 0 to about 2000 ppm NaCl. Normal growth at NaCl concentrations of about 500 ppm or more means that the acetogenic bacteria includes a sodium-translocating ATPase. In this aspect, the composition of the fermentation medium also includes a sodium ion concentration of about 40 to about 500 mmol per liter, in another aspect, about 40 to about 250 mmol per liter and in another aspect, a sodium ion concentration of about 50 to about 200 mmol per liter. In one aspect, the sodium ion concentration is about 500 ppm to about 8000 ppm, in another aspect, about 1000 ppm to about 7000 ppm, in another aspect, about 3000 ppm to about 6000 ppm, in another aspect, about 2000 to about 5000 ppm, and in another aspect, about 3000 to about 4000 ppm.

Examples of useful acetogenic bacteria for CO₂ bioconversion include Acetobacterium bacteria, Acetogenium kivui, Acetoanaerobium noterae, Acetobacterium woodii, Acetobacterium bakii Alkalibaculum bacchi CP11 (ATCC BAA-1772), Moorella thermoacetica, Moorella thermoautotrophica, Ruminococcus productus, Acetogenium kivui, and combinations thereof.

Fermentable gaseous substrate refers to C1-containing gaseous substrate comprises one or more of CO, CO₂, or CH₂O₂. Suitable gaseous substrate may include various synthesis gas (i.e. syngas) and industrial off-gas.

Syngas may be provided from any known source. In one aspect, syngas may be sourced from gasification of carbonaceous materials. Gasification involves partial combustion of carbonaceous materials feedstock in a restricted supply of oxygen. The resultant gas may include CO, CO₂, and H₂. A negligible amount of oxygen (<1,000 ppm) may be present in the gasification resultant gas. Some examples of suitable gasification methods and apparatus are provided in U.S. Ser. Nos. 61/516,667, 61/516,704 and 61/516,646, all of which were filed on Apr. 6, 2011, and in U.S. Ser. Nos. 13/427,144, 13/427,193 and 13/427,247, all of which were filed on Mar. 22, 2012, and all of which are incorporated herein by reference. In another aspect, syngas may be generated from electrolysis of water and carbon dioxide. In this aspect, oxygen is removed from the resultant gas and the resultant gas may be further blended with other gas sources to form a desired fermentable C1-containing gaseous substrate.

The syngas produced from gasification of carbonaceous materials may contain low carbon monoxide concentration due to operating condition variance, gasifier design and low calorific value of the carbonaceous materials. Carbonaceous materials with low calorific value contain 13 MJ/kg or less calorific value, in one aspect, 12 MJ/kg or less, in one aspect, 10 MJ/kg or less, in one aspect 9 MJ/kg to 13 MJ/kg, in one aspect 8 MJ/kg to 13 MJ/kg, and in another aspect, 7 MJ/kg to 12 MJ/kg. Examples of carbonaceous materials with low calorific value include, but not limited to, feedstocks containing 20% or more moisture content and municipal solid waste (MSW). In this aspect, the syngas with low carbon monoxide concentration contains 30% or less CO, in another aspect, 25% or less CO, in another aspect, 20% or less CO, in another aspect, 15% or less CO, in another aspect, 10% to 30% CO, in another aspect, 10% to 25% CO, and in another aspect, 5% to 30% CO. Meanwhile, gasifying low calorific value carbonaceous materials also produces significantly more carbon dioxide in the syngas. The syngas with low carbon monoxide concentration may contain 20% or more CO₂, in another aspect, 25% or more CO₂, in another aspect, 30% or more CO₂, in another aspect, 20% to 30% CO₂, in another aspect, 25% to 40% CO₂, in another aspect, 20% to 40% CO₂, and in another aspect, 30% to 50% CO₂. Fermenting syngas with low carbon monoxide concentration and high carbon dioxide concentration in CO bioconversion process may result low target oxygenated hydrocarbonaceous compounds production and cause operating issues, such as foaming, since the CO converting anaerobic bacteria may not be able to utilize CO₂ in the syngas. CO₂ in the syngas with low carbon monoxide concentration may then be separated out and sent to the subsequent CO₂ bioconversion process to be fermented by the CO₂ converting anaerobic bacteria. Meanwhile, the CO₂ may also be sent to a CO₂ electrolyzer to be electrolyzed into CO and O₂. The electrolyzer generated CO is then provided to the CO conversion bioreactor to increase oxygenated hydrocarboncaceous compounds production and the electrolyzer generated O₂ is provided to the gasifier to increase the efficiency of the gasification process. After the removal of the CO₂ from the syngas with low carbon monoxide concentration, CO concentration in the syngas increases. The CO concentrated syngas may then be directly fermented by the CO converting anaerobic bacteria in the CO conversion bioreactor.

By contrast, the syngas produced from gasification of carbonaceous materials with high calorific value may be directly fermentable by the CO converting anaerobic bacteria in the CO conversion bioreactor. In this aspect, the syngas contains 30% or more CO, in one aspect, 35% or more CO, in one aspect, 37% or more CO, in another aspect, 40% or more CO. Meanwhile, such syngas contains 20% or less CO₂, in one aspect, 18% or less CO₂, in another aspect, 15% or less CO₂, in another aspect, 12% or less CO₂, and still in another aspect, 10% or less CO₂. Carbonaceous materials with high calorific value contain more than 13 MJ/kg calorific value, in one aspect, more than 14 MJ/kg, in one aspect, 15 MJ/kg or more, in one aspect 13.5 to 18 MJ/kg, in one aspect, 14 to 30 MJ/kg, and in another aspect, 14 to 22 MJ/kg. Examples of carbonaceous materials with high calorific value include, but not limited to dried wood chips, switchgrass, corn cobs and corn stover.

Industrial off-gas may include the C1-containing waste gas from industrial processes that would otherwise be exhausted into the atmosphere. Examples of industrial off-gases include gases produced during microbial fermentation, ferrous metal products manufacturing, non-ferrous products manufacturing, petroleum refining processes, gasification of coal, electric power production, carbon black production, ammonia production, methanol production, coke manufacturing and gas reforming. In one aspect, the industrial off-gas may be a flue gas from incineration of biogenic waste materials, such as municipal solid waste (MSW), medical waste, and/or hazardous waste. The incineration flue gas may include 10% to 20% CO₂, 60% to 75% N₂, and 3% to 5% O₂. Since incineration typically operates at high temperatures (800° C. to 1200° C.) and at excessive air/oxygen levels with a complete combustion of waste materials, the CO concentration in the flue gas may be negligible (<1,000 ppm). The incineration flue gas may be fermented by the CO₂ converting anaerobic bacteria. In this aspect, O₂ is removed before the incineration flue gas is provided to the CO₂ conversion bioreactor.

A CO₂ electrolyzer may be used to electrolyze external CO₂ gas or CO₂ gas generated during the bioconversion process into CO and O₂ at controlled temperature (such as above 600° C.): 2CO₂+Electricity=2CO+O₂. CO gas is collected at the cathode, while O₂ gas is collected at the anode. Water vapor may also be added along with the CO₂ gas into the CO₂ electrolyzer to generate H₂ and additional O₂. In such case, H₂ and CO gas are collected at the cathode and O₂ gas is collected at the anode. In one aspect, the CO generated is supplemented into a CO-containing gaseous substrate and fermented by a CO converting anaerobic bacteria in a CO conversion bioreactor. In another aspect, the CO and H₂ generated are supplemented into a CO-containing gaseous substrate and fermented by a CO converting anaerobic bacteria in a CO conversion bioreactor. In another aspect, the H₂ generated is supplemented into a CO₂-containing gaseous substrate and fermented by a CO₂ converting anaerobic bacteria in a CO₂ conversion bioreactor. The O₂ produced may further be provided to a gasifier and/or an incinerator or be vented.

The C1-containing gaseous substrate may include H₂. H₂ may also be separately supplemented into the C1-containing gaseous substrate to form desired gas composition suitable for fermentation. Examples of H₂ source include gases produced during ferrous metal products manufacturing, non-ferrous products manufacturing, petroleum refining processes, gasification of coal, gasification of biomass, electric power production, carbon black production, ammonia production, methanol production and coke manufacturing. Other sources of hydrogen may include for example, H₂O electrolysis and bio-generated H₂. In one aspect, a water electrolyzer is used to electrolyze liquid phase water into H₂ and O₂ at 25 to 100° C.: 2H₂O+Electricity=2H₂+O₂. H₂ gas is collected at the cathode, while O₂ gas is collected at the anode. The H₂ generated may then be supplemented to one or more bioreactors and the O₂ generated may be further provided to a gasifier and/or an incinerator or be vented. In one aspect, the H₂ generated is supplemented into a CO₂-containing gaseous substrate and fermented by a CO₂ converting anaerobic bacteria in a CO₂ conversion bioreactor. In another aspect, the H₂ generated is supplemented into a CO-containing gaseous substrate and fermented by a CO converting anaerobic bacteria in a CO conversion bioreactor. In another aspect, the H₂ generated is supplemented to both a CO conversion bioreactor and a CO₂ conversion bioreactor.

In one aspect, at least a portion of the electricity provided to the CO₂ electrolyzer and/or the water electrolyzer is renewable electricity generated from a power source with renewable power, such as wind power, solar power, hydro power, tidal power, and geothermal power. Heat generated from the gasifier or incinerator can also be used as a power source. In one aspect, a portion of the electricity used by electrolyzer is directly generated by the heat and/or steam from the gasifier or incinerator.

The bioconversion process may include two or more bioreactors to conduct both CO bioconversion and CO₂ bioconversion. For example, the fermentation process may contain one or more CO conversion bioreactor with CO converting anaerobic bacteria and one or more CO₂ conversion bioreactor with CO₂ converting anaerobic bacteria. In this aspect, the CO converting anaerobic bacteria and the CO₂ converting anaerobic bacteria are different species or strains. CO₂ can be a byproduct of CO bioconversion process. Vent gas from CO bioconversion contains CO₂ and can be used in one or more subsequent CO₂ conversion bioreactors as a CO₂-containing gaseous substrate. One or more fermentation liquid broth streams are purged out of the two or more bioreactors and separated into one or more cell free permeates and cell-containing suspensions through one or more cell separators. One or more valuable oxygenated hydrocarbonaceous compounds can later be recovered from the one or more cell free permeates. Meanwhile, the permeate containing organic acids produced in the CO₂ bioconversion process is sent to the one or more CO conversion bioreactors and the said organic acid may be fermented by the CO converting anaerobic bacteria into the one or more alcohols in presence of carbon monoxide. In one aspect, the organic acid produced in the CO₂ bioconversion is acetic acid. In this aspect, the acetic acid-containing permeate is further delivered to the one or more CO conversion bioreactors and acetic acid is subsequently converted into alcohol by the CO converting anaerobic bacteria. In one aspect, a CO₂ electrolyzer is configured to receive at least a portion of the CO₂ contained in the vent gas from the CO conversion bioreactor to balance the CO₂ consumption of the CO₂ conversion bioreactor and the organic acid consumption of the CO conversion bioreactor. In this aspect, a CO₂ separator is placed to receive the vent gas from the CO conversion bioreactor and provide a CO₂ concentrated vent gas to the CO₂ electrolyzer and a CO₂ diluted vent gas to the CO₂ conversion bioreactor. In one aspect, the CO₂ concentrated vent gas contains 50% or less of the CO₂ generated by the CO conversion bioreactor, in another aspect, 40% or less of the CO₂ generated by the CO conversion bioreactor, in another aspect, 30% or less of the CO₂ generated by the CO conversion bioreactor, in another aspect, 20% or less of the CO₂ generated by the CO conversion bioreactor, in another aspect, 10% or less of the CO₂ generated by the CO conversion bioreactor, in another aspect, 15% to 40% of the CO₂ generated by the CO conversion bioreactor, in another aspect, 10% to 35% of the CO₂ generated by the CO conversion bioreactor, in another aspect, 10% to 30% of the CO₂ generated by the CO conversion bioreactor, and in still another aspect, 5% to 40% of the CO₂ generated by the CO conversion bioreactor. In another aspect, the CO₂ electrolyzer is configured to receive at least a portion of the CO₂ contained in the exhaust gas from the CO₂ conversion bioreactor. In this aspect, a CO₂ separator is placed to receive the exhaust gas from the CO₂ conversion bioreactor and provide a CO₂ concentrated gas stream to the CO₂ electrolyzer and a CO₂ diluted exhaust gas. In one aspect, the CO₂ diluted exhaust gas contains 5% or less CO₂, in another aspect, 3% or less CO₂, in another aspect 1% or less CO₂, and in another aspect, 0.5% or less CO₂. The CO₂ diluted exhaust gas may then be passed to a CO₂ removal unit to remove all of the CO₂ and generate a CO₂ free exhaust gas. Suitable CO₂ removal unit includes, but not limited to, scrubbers, filters, methyl diethanolamine system (MDEA), Rectisol system, and the combination thereof. In still another aspect, both the at least a portion of the vent gas from the CO conversion bioreactor and the at least a portion of the exhaust gas from the CO₂ conversion bioreactor are provided to the CO₂ electrolyzer.

The term reduced carbon dioxide emission means most of the carbon in the CO-containing gaseous substrate provided to the bioconversion process is converted into oxygenated hydrocarbonaceous compounds and single cell protein, and the process releases significantly lower amount of CO₂ to the atmosphere compared to a conventional CO bioconversion fermentation process. In the bioconversion process with one or more CO conversion bioreactors and one or more CO₂ conversion bioreactors, CO₂ is a byproduct from the CO conversion bioreactor and is sent to a CO₂ conversion bioreactor for CO₂ bioconversion fermentation. The CO₂ conversion bioreactor may convert 70% to 100% of the CO₂ sent to the bioreactor, in one aspect, 75% to 100% of the CO₂ sent to the bioreactor, in one aspect, 85% to 100% of the CO₂ sent to the bioreactor, in one aspect, 90% to 100% of the CO₂ sent to the bioreactor, and in one aspect, 95% to 100% of the CO₂ sent to the bioreactor. An organic acid-containing permeate is produced from the CO₂ conversion bioreactor and is sent to the CO conversion bioreactor. The CO converting bacteria is capable of converting the organic acid into oxygenated hydrocarbonaceous compounds in presence of carbon monoxide. In this aspect, the bioconversion process with at least one CO conversion bioreactor and at least one CO₂ conversion bioreactor can achieve at least 80% of CO conversion, in another aspect, at least 90% of CO conversion, in another aspect, at least 95% of CO conversion, in another aspect, at least of 99% of CO conversion, and in still another aspect, 100% of CO conversion is achieved. The exhaust gas from the CO₂ conversion bioreactor contains 15% or less CO₂, in one aspect, 10% or less CO₂, in one aspect, 5% or less CO₂, in one aspect, 3% or less CO₂, in one aspect, 1% or less CO₂, in one aspect, 3% to 15% CO₂, in one aspect, 3% to 10% CO₂, in one aspect, 1% to 10% CO₂, in one aspect, 1% to 5% CO₂, and in another aspect, the exhaust gas contains no CO₂. In the scenario that the exhaust gas from the CO₂ conversion bioreactor contains CO₂, the exhaust gas may be further sent to a CO₂ separator to generate a CO₂ concentrated gas and a CO₂ free exhaust gas. The CO₂ concentrated gas may be recycled back to the CO₂ conversion bioreactor or sent to a CO₂ electrolyzer. Selection could be made by comparing the cost of the H₂ consumed to convert the CO₂ in the CO₂ conversion bioreactor and the cost of the electricity consumed to electrolyze the CO₂ by the CO₂ electrolyzer. In this scenario, the whole system achieves zero carbon dioxide emission. The exhaust gas may also contain 3% or less CO, in one aspect, 2% or less CO, in one aspect, 1% or less CO, in another aspect, 0.5% or less CO, and in another aspect, the exhaust gas contains no CO. In this scenario, at least 85% of the carbon from carbon monoxide and carbon dioxide in the CO-containing gaseous substrate provided to the bioconversion process has been fixed/sunk into organic hydrocarbonaceous compounds and single cell protein, in one aspect, at least 87% of the carbon from carbon monoxide and carbon dioxide provided, in one aspect, at least 90% of the carbon from carbon monoxide and carbon dioxide provided, in another aspect, at least 93% of the carbon from carbon monoxide and carbon dioxide provided, in another aspect, at least 95% of the carbon from carbon monoxide and carbon dioxide provided, and in still another aspect, at least 99% of the carbon from carbon monoxide and carbon dioxide provided.

The term negative carbon dioxide emission is also known as carbon dioxide removal, which means a system releases less CO₂ to the atmosphere than the CO₂ it takes in from an external source. External CO₂ may come from an industrial off-gas or an incineration flue gas or a syngas and be provided to the bioconversion process with one or more CO₂ conversion bioreactors as CO₂-containing gaseous substrate. In this aspect, the CO₂-containing gaseous substrate can be fixed/sunk into organic hydrocarbonaceous compounds and single cell protein.

The fermentation process of the underlying disclosure provides a simultaneous approach of generating a high specific productivity of oxygenated hydrocarbon compound production while producing nutrient supplement from the bacterial cells used in the fermentation process. As used herein, specific productivity is expressed as specific STY. In this aspect, specific oxygenated hydrocarbon compound productivity may be expressed as specific STY (e.g. specific space time yield can be expressed as grams alcohol/day/gram of cells or grams organic acid/day/gram of cells). In one aspect, the fermentation process provides a specific organic acid productivity of about 0.2 to about 50 grams organic acid/day/gram of cells, in another aspect, about 0.2 to about 20 grams organic acid/day/gram of cells, in another aspect, about 10 to about 50 grams organic acid/day/gram of cells, in another aspect, about 14 to about 30 grams organic acid/day/gram of cells, in another aspect, about 2 to about 20 grams organic acid/day/gram of cells, and in another aspect, about 15 to about 25 grams organic acid/day/gram of cells. In this aspect, the organic acid is acetic acid or butyric acid, or a mixture of both. In another aspect, the fermentation process provides a specific alcohol productivity of about 10 grams alcohol/day/grams of cells or more, in another aspect, a specific alcohol productivity rate of about 12 g/day/grams of cells or more, in another aspect, a specific alcohol productivity rate of about 14 g/day/grams of cells or more, in another aspect, a specific alcohol productivity rate of about 10 to about 16 g/day/grams of cells, in another aspect, about 10 to about 14 g/day/grams of cells, in another aspect, about 10 to about 12 g/day/grams of cells, in another aspect, about 10 to about 16 g/day/grams of cells, in another aspect, about 10 to about 14 g/day/grams of cells, in another aspect, about 12 to about 16 g/day/grams of cells, and in another aspect, about 12 to about 14 g/day/grams of cells. In this aspect, the alcohol is ethanol or butanol, or a mixture of both. In the scenario that the C1-containing gaseous substrate is a syngas produced from a gasification process, the overall process produces 220 kg or more alcohol per metric ton carbonaceous material gasified in the gasifier, in another aspect, 250 kg or more alcohol per metric ton carbonaceous material gasified in the gasifier, in another aspect, 300 kg or more alcohol per metric ton carbonaceous material gasified in the gasifier, and still in another aspect, 350 kg or more alcohol per metric ton carbonaceous material gasified in the gasifier.

Further, the fermentation process can be manipulated under conditions that facilitate the production of a desired product. In one aspect, the desired product is one or more oxygenated hydrocarbonaceous compounds. In another aspect, the desired product is the microbial biomass, and the process also produces other oxygenated hydrocarbonaceous compounds as byproducts. Operation parameters, such as culture medium flow rate, gaseous substrate feed rate, water supply/recycle rate, temperature, media redox potential, pressure, pH, agitation rate (if using a stirred tank reactor), and cell concentration, are monitored and controlled throughout the fermentation process.

A fermentation liquid broth is generated inside the bioreactor once the fermentation process is started. In addition to the culture medium, the fermentation liquid broth also includes acetogenic bacteria and one or more oxygenated hydrocarbon compounds. In one aspect, the cell concentration of the fermentation liquid broth is about 1 to about 15 g/L, in another aspect 5 to about 30 g/L, in another aspect, about 10 to about 25 g/L, in another aspect, about 8 to about 20 g/L, in another aspect, about 9 to about 15 g/L, and in another aspect, about 10 to about 30 g/L.

The fermentation liquid broth is further purged out of the bioreactor and then separated into a cell free permeate and a cell-containing suspension by one or more cell separators. Suitable cell separators include, but are not limited to, filtration devices, hollow fiber filtration devices, spiral wound filtration devices, ultrafiltration devices, ceramic filter devices, cross-flow filtration devices, size exclusion column filtration devices, spiral wound membranes, centrifugation devices, and combination thereof.

The cell free permeate contains one or more desired oxygenated hydrocarbonaceous compounds and is sent to a distillation system for product recovery. One or more desired products are recovered and collected from the distillation system. In one aspect, a holding tank is placed between the one or more cell separators and the distillation system to receive the cell free permeate and control cell free permeate flow rate to the distillation system. The distillation bottoms may be recycled back to the bioreactor. In one aspect, at least a portion of the distillation bottoms is recycled back to the bioreactor. In another aspect, at least a portion of the distillation bottoms is sent to a wastewater treatment system for further treatment. In still another aspect, at least a portion of the distillation bottoms is recycled back to the bioreactor and at least a portion of the distillation bottoms is sent to a wastewater treatment system.

The cell-containing suspension contains acetogenic bacterial cells at a cell concentration higher than the fermentation liquid broth. In one aspect, the cell concentration of the cell-containing suspension is about 20 g/L or more, in another aspect, about 30 g/L or more, in another aspect, about 40 g/L or more, in another aspect, about 50 g/L or more, in another aspect, about 60 g/L or more, in another aspect, about 20 to about 300 g/L, in another aspect, about 30 to about 250 g/L, in another aspect, about 40 to about 200 g/L, in another aspect, about 50 to about 150 g/L, in still another aspect, about 100 to about 150 g/L. At least a portion of the cell-containing suspension may be recycled back to the bioreactor to maintain and control the cell concentration in the fermentation process. Additional cell-containing suspension can also be further processed into nutrient supplement. In one aspect, at least a portion of the cell-containing suspension is recycled back to the bioreactor. In another aspect, at least a portion of the cell-containing suspension is further processed to nutrient supplement. In still another aspect, at least a portion of the cell-containing suspension is recycled back to the bioreactor and at least a portion of the cell-containing suspension is further processed to nutrient supplement.

Multiple cell separators may be used in the fermentation process to adjust and balance the production of the oxygenated hydrocarbonaceous compound and the nutrient supplement so that the desired productivity of the oxygenated hydrocarbonaceous compound is maintained while at least some of the bacterial cells are recovered into nutrient supplement. In one aspect, at least two cell separators are coupled with one bioreactor. In this aspect, a first fermentation liquid broth is sent to a first cell separator to produce a first cell free permeate and a first cell-containing suspension. The first cell free permeate is further sent to a distillation system for the recovery of the oxygenated hydrocarbonaceous compound and at least a portion of the first cell-containing suspension is recycled back to the bioreactor to control and maintain cell concentration of the fermentation liquid broth. Further, a second fermentation liquid broth is sent to a second cell separator to produce a second cell free permeate and a second cell-containing suspension. The second cell free permeate is sent to the distillation system for the recovery of the oxygenated hydrocarbonaceous compound and the second cell-containing suspension is then processed into nutrient supplement. In another aspect, three or more cell separators are used in a multi-bioreactor fermentation process.

Systems for Producing Oxygenated Hydrocarbonaceous Compound with Reduced or Negative Carbon Dioxide Emissions.

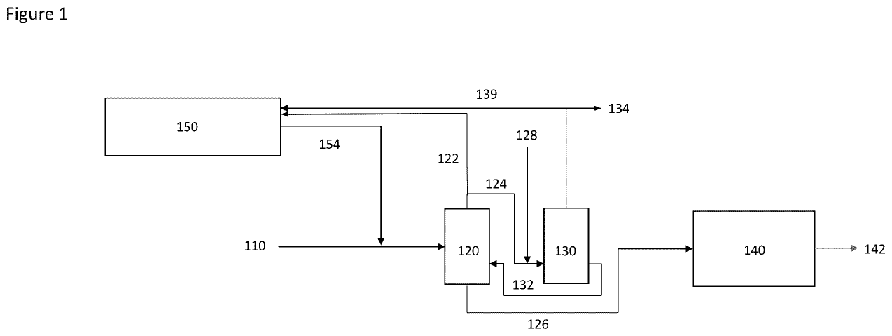

FIG. 1 illustrates a schematic of a system for producing an oxygenated hydrocarbonaceous compound with reduced carbon dioxide emissions. The system includes a CO conversion bioreactor 120, a CO₂ conversion bioreactor 130, a distillation system 140, and a CO₂ electrolyzer 150.

A CO-containing gaseous substrate 110 is provided to the CO conversion bioreactor 120. In the CO conversion bioreactor 120, CO is fermented by a CO converting anaerobic bacteria into one or more oxygenated hydrocarbonaceous compounds and CO₂. A cell free oxygenated hydrocarbonaceous compound containing permeate 126 is then delivered to the distillation system 140 to recover an oxygenated hydrocarbonaceous compound 142. The CO₂-containing vent gas 124 from the CO conversion bioreactor 120 is sent to the CO₂ conversion bioreactor 130. In the CO₂ conversion bioreactor 130, CO₂ is fermented by a CO₂ converting anaerobic bacteria into one or more organic acids. Additional H₂ and/or external CO₂ stream 128 may be blended with the CO₂-containing vent gas 124 to form a desired H₂:CO₂ ratio for the CO₂ bioconversion. The external CO₂ stream 128 may be collected from an industrial off-gas. A cell free organic acid-containing permeate 132 is delivered to the CO conversion bioreactor 120. The CO converting anaerobic bacteria in the CO conversion bioreactor 120 is capable of converting the one or more organic acids into the one or more oxygenated hydrocarbonaceous compounds in presence of carbon monoxide. At least a portion of the CO₂ 122 of the CO₂-containing vent gas 124 is sent to a CO₂ electrolyzer 150 to produce a CO stream 154. The amount of the CO₂ 122 sent to the CO₂ electrolyzer 150 is based on the balance of the CO₂ conversion rate of the CO₂ conversion bioreactor 130 and the organic acid conversion rate of the CO conversion bioreactor 120. The CO stream 154 may be then directly sent to the CO conversion bioreactor 120 or be mixed with the CO-containing gaseous substrate 110. The CO₂ conversion bioreactor further generates an exhaust gas 134. The exhaust gas 134 may contain a low amount of CO₂. In this scenario, the CO₂ 139 from the exhaust gas 134 may then be recycled back to the CO₂ conversion bioreactor 130 (not shown in FIG. 1) or to the CO₂ electrolyzer 150 to achieve zero carbon dioxide emission. In another scenario, the CO₂ 139 from the exhaust gas 134 is removed through a scrubber, a methyl diethanolamine system, a Rectisol system or a filtration system.

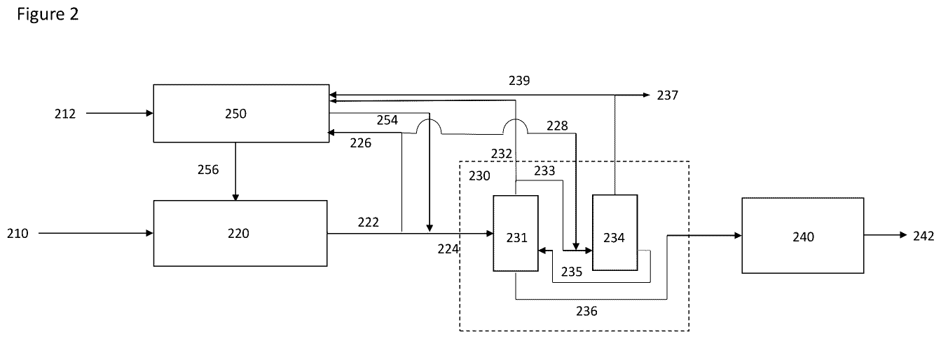

FIG. 2 illustrates a schematic of a system for producing an oxygenated hydrocarbonaceous compound with reduced or negative carbon dioxide emissions. The system includes a gasifier 220, a bioconversion system 230, a distillation system 240, and a CO₂ electrolyzer 250.

A carbonaceous materials feedstock 210 is fed to a gasifier 220 to produce a CO-containing syngas 222. In one scenario, the CO-containing syngas 222 is fermentable and can be directly sent to a bioconversion system 230. The bioconversion system 230 contains at least one CO conversion bioreactor 231 and one CO₂ conversion bioreactor 234. The CO conversion bioreactor 231 receives the CO-containing syngas 222 and ferments the syngas with a CO converting anaerobic bacteria to produce a cell free oxygenated hydrocarbonaceous compounds containing permeate 236 and a CO₂-containing vent gas 233. In another scenario, directly fermenting the CO-containing syngas 222 by the CO conversion bioreactor 231 may not be preferred due to low CO concentration. In this scenario, the CO₂ contained in the CO-containing syngas 222 can be separated out to generate to a CO-concentrated fermentable syngas 224. The CO-concentrated fermentable syngas 224 is delivered to the CO conversion bioreactor 231. In one aspect, at least a portion of the CO₂ 226 separated from the CO-containing gas 222 is sent to the CO₂ electrolyzer 250. In another aspect, at least a portion of the CO₂ 228 separated from the CO-containing gas 222 is sent to the CO₂ conversion bioreactor 234. In another aspect, the CO₂ 226 is sent to the CO₂ electrolyzer 250 and the CO₂ 228 is sent to the CO₂ conversion bioreactor 234. A distillation system 240 is configured to receive the cell free oxygenated hydrocarbonaceous compounds permeate 236 and recover one or more oxygenated hydrocarbonaceous compounds 242. The CO₂-containing vent gas 233 is then sent to the CO₂ conversion bioreactor 234. The CO₂-containing vent gas 233 may be blended with a supplemental H₂ gas and/or at least a portion of the CO₂ 228 separated from the CO-containing gas 222 to form a desired H₂ to CO₂ ratio before sending to the CO₂ conversion bioreactor 234. The CO₂ conversion bioreactor 234 ferments the CO₂-containing vent gas 233 with a CO₂ converting anaerobic bacteria to produce a cell free organic acids-containing permeate 235 with one or more organic acids and an exhaust gas 237. The cell free organic acids containing permeate 235 is recycled to the CO conversion bioreactor 231. The CO converting anaerobic bacteria in the CO conversion bioreactor 231 is capable of converting the one or more organic acids into the one or more oxygenated hydrocarbonaceous compounds in presence of carbon monoxide.

CO₂ from the bioconversion system 230 can be sent to the CO₂ electrolyzer 250. In one scenario, at least a portion of the CO₂ 232 in the CO₂-containing vent gas 233 is sent to the CO₂ electrolyzer 250. In another scenario, the exhaust gas 237 may contain a low amount of CO₂. The CO₂ 239 from the exhaust gas 237 is recycled back to the CO₂ conversion bioreactor 234 or the CO₂ electrolyzer 250. In still another scenario, both the CO₂ 232 and the CO₂ 239 are sent to the CO₂ electrolyzer 250. Selection could be made by comparing the cost of the H₂ consumed to convert the CO₂ in the CO₂ conversion bioreactor 234 and the cost of the electricity consumed to electrolyze the CO₂ by the CO₂ electrolyzer 250. The CO₂ electrolyzer 250 may also receive an external CO₂ stream 212, which may be collected from an industrial off-gas. The CO₂ electrolyzer 250 further produces a CO stream 254 and an O₂ stream 256. The CO stream 254 may be directly sent to the CO conversion bioreactor 231, be mixed with the CO-containing syngas 222 or be mixed with the CO-concentrated fermentable syngas 224. The O₂ stream 256 is provided to the gasifier 220 as oxidant agent.

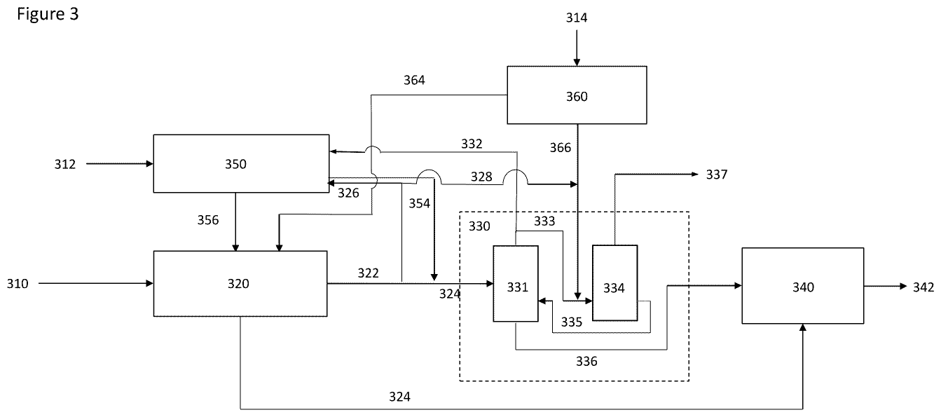

FIG. 3 illustrates a schematic of a system for generating power and producing an oxygenated hydrocarbonaceous compound with reduced or negative carbon dioxide emissions. The system includes a gasifier 320, a bioconversion system 330, a distillation system 340, a CO₂ electrolyzer 350, and a H₂O electrolyzer 360.

A carbonaceous materials feedstock 310 is fed to a gasifier 320 to produce a CO-containing syngas 322 and a steam 324. In one scenario, the CO-containing syngas 322 is fermentable and can be directly sent to a bioconversion system 330. The bioconversion system 330 contains at least one CO conversion bioreactor 331 and one CO₂ conversion bioreactor 334. The CO conversion bioreactor 331 receives the CO-containing fermentable syngas 322 and ferments the syngas with a CO converting anaerobic bacteria to produce a cell free oxygenated hydrocarbonaceous compounds containing permeate 336 and a CO₂-containing vent gas 333. In another scenario, directly fermenting the CO-containing syngas 322 by the CO conversion bioreactor 331 may not be preferred due to low CO concentration. In this scenario, the CO₂ contained in the CO-containing syngas 322 can be separated out to generate to a CO-concentrated fermentable syngas 324. The CO-concentrated fermentable syngas 324 is delivered to the CO conversion bioreactor 331. In one aspect, at least a portion of the CO₂ 326 separated from the CO-containing gas 322 is sent to the CO₂ electrolyzer 350. In another aspect, at least a portion of the CO₂ 328 separated from the CO-containing gas 322 is sent to the CO₂ conversion bioreactor 334. In another aspect, the CO₂ 326 is sent to the electrolyzer 350 and the CO₂ 328 is sent to the CO₂ conversion bioreactor 334. Selection could be made by comparing the cost of the H₂ consumed to convert the CO₂ in the CO₂ conversion bioreactor 334 and the cost of the electricity consumed to electrolyze the CO₂ by the CO₂ electrolyzer 350. A distillation system 340 is configured to receive cell free oxygenated hydrocarbonaceous compounds permeate 336 and recover one or more oxygenated hydrocarbonaceous compounds 342. The steam 324 is provided to the distillation system 340 as a heat source. The CO₂-containing vent gas 333 is then sent to the CO₂ conversion bioreactor 334. The CO₂-containing vent gas 333 may be blended with a supplemental H₂ gas and/or the CO₂ 328 separated from the CO-containing gas 322 to form a desired H₂ to CO₂ ratio before sending to the CO₂ conversion bioreactor 334. The CO₂ conversion bioreactor 334 ferments the CO₂-containing vent gas 333 with a CO₂ converting anaerobic bacteria to produce a cell free organic acids containing permeate 335 with one or more organic acids and an exhaust gas 337. The cell free organic acids containing permeate 335 is recycled to the CO conversion bioreactor 331. The CO converting anaerobic bacteria in the CO conversion bioreactor 331 is capable of converting the one or more organic acids into the one or more oxygenated hydrocarbonaceous compounds in presence of carbon monoxide.

CO₂ from the bioconversion system 330 can be sent to the CO₂ electrolyzer 350. In one scenario, at least a portion of the CO₂ 332 in the CO₂-containing vent gas 333 is sent to the CO₂ electrolyzer 350. In another scenario, the exhaust gas 337 may contain a low amount of CO₂. The CO₂ from the exhaust gas 337 is then recycled back to the CO₂ conversion bioreactor 334 or the CO₂ electrolyzer 350. In still another scenario, both the CO₂ 332 and the CO₂ contained in the exhaust gas 337 are sent to the electrolyzer 350. The CO₂ electrolyzer 350 may also receive an external CO₂ stream 312, which may be collected from an industrial off-gas. The CO₂ electrolyzer 350 further produces a CO stream 354 and a first O₂ stream 356. The CO stream 354 may be directly sent to the CO conversion bioreactor 331, be mixed with the CO-containing syngas 322, or be mixed with the CO-concentrated fermentable syngas 324. A H₂O electrolyzer 360 is configured to receive a water stream 314 and produce a H₂ stream 366 and a second O₂ stream 364. The H₂ stream 366 may be used as a source of additional H₂ and blended with the CO₂-containing vent gas 333 and/or the CO₂ 328 from the CO-containing syngas 322 to form a desired H₂:CO₂ ratio for the CO₂ bioconversion. Both the first O₂ stream 356 and the second O₂ stream 364 are provided to the gasifier 320 as oxidant agents.

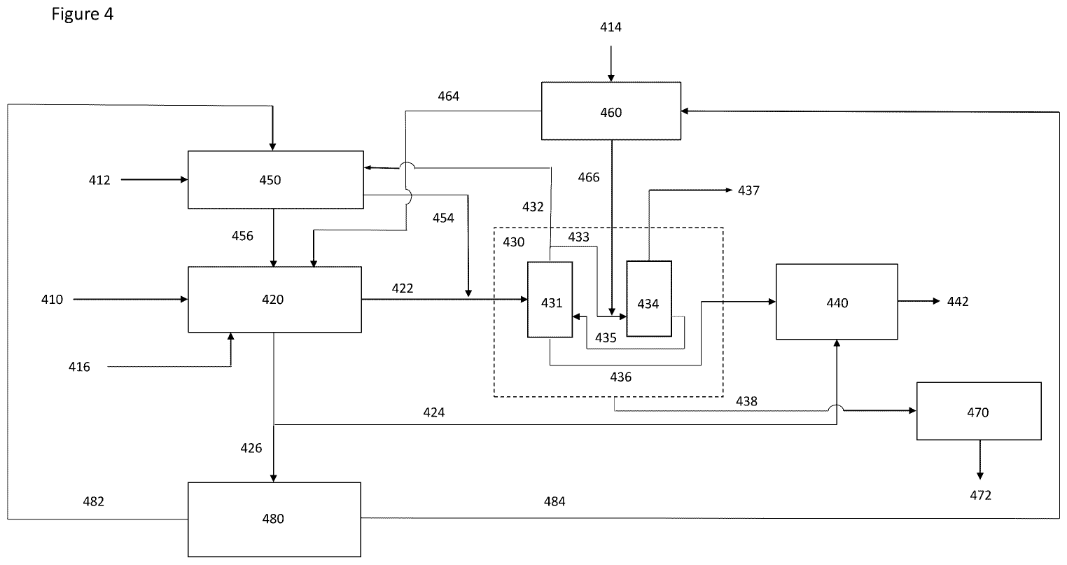

FIG. 4 illustrates a schematic of a system for generating power and producing an oxygenated hydrocarbonaceous compound and a single cell protein with reduced or negative carbon dioxide emissions. The system includes a gasifier 420, a bioconversion system 430, a distillation system 440, a CO₂ electrolyzer 450, a H₂O electrolyzer 460, a cell mass processing unit 470, and a renewable power source 480.

A biomass feedstock 410 is fed to a gasifier 420 to produce a CO-containing syngas 422 and steam 424 and 426. The CO-containing syngas 422 is sent to a bioconversion system 430. The bioconversion system 430 contains at least one CO conversion bioreactor 431 and one CO₂ conversion bioreactor 434. The CO conversion bioreactor 431 receives the CO-containing syngas 422 and ferments the syngas with a CO converting anaerobic bacteria to produce a cell free oxygenated hydrocarbonaceous compounds containing permeate 436 and a CO₂-containing vent gas 433. A distillation system 440 is configured to receive the cell-free oxygenated hydrocarbonaceous compounds containing permeate 436 and recover one or more oxygenated hydrocarbonaceous compounds 442. At least a portion of the steam 424 is provided to the distillation system 440 as a heat source. The CO₂-containing vent gas 433 is then sent to the CO₂ conversion bioreactor 434. The CO₂ conversion bioreactor 434 ferments the CO₂-containing vent gas 433 with a CO₂ converting anaerobic bacteria to produce a cell free organic acids containing permeate 435 with one or more organic acids and an exhaust gas 437. Additional H₂ may be blended with the CO₂-containing vent gas 433 to form a desired H₂:CO₂ ratio for the CO₂ bioconversion. The cell free organic acids containing permeate 435 is recycled to the CO conversion bioreactor 431. The CO converting anaerobic bacteria in the CO conversion bioreactor 431 is capable of converting the one or more organic acids into the one or more oxygenated hydrocarbonaceous compounds in presence of carbon monoxide. Both the CO conversion bioreactor 431 and the CO₂ conversion bioreactor 434 continuously purge out cell mass during operation. Multiple cell separators and/or holding tanks are configured to concentrate the cell mass and produce a cell mass containing suspension 438. The cell mass containing suspension is then sent to the cell mass processing unit 470 to be processed into single cell protein 472.

The exhaust gas 437 may contain a low amount of CO₂. The CO₂ from the exhaust gas 437 is then recycled back to the CO₂ conversion bioreactor 434 or a CO₂ electrolyzer 450. At least a portion of the CO₂ 432 in the CO₂-containing vent gas 433 may be sent to the CO₂ electrolyzer 450. The CO₂ electrolyzer 450 may also receive an external CO₂ stream 412, which may be collected from an industrial off-gas. The CO₂ electrolyzer 450 further produces a CO stream 454 and a first O₂ stream 456. The CO stream 454 may be directly sent to the CO conversion bioreactor 431 or be mixed with the CO-containing fermentable syngas 422. A H₂O electrolyzer 460 is configured to receive a water stream 414 and produce a H₂ stream 466 and a second O₂ stream 464. The H₂ stream 466 may be used as a source of additional H₂ and blended with the CO₂-containing vent gas 433 to form a desired H₂:CO₂ ratio for the CO₂ bioconversion. Both the first O₂ stream 456 and the second O₂ stream 464 are provided to the gasifier 420 as oxidant agents. Other oxidant agent 416, such as air, steam and CO₂, may also be added to the gasifier 420. At least another portion of the steam 426 generated from the gasifier 420 is provided to the renewable power source 480. The renewable power source produces renewable electricity 482 and 484, which are used as power source for the CO₂ electrolyzer 450 and the H₂ electrolyzer 460.

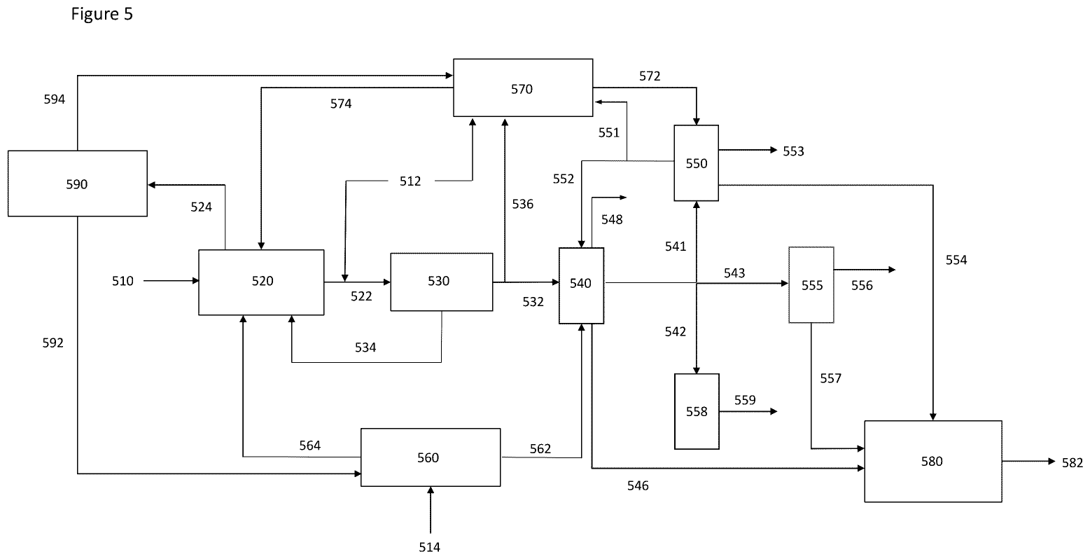

FIG. 5 illustrates a schematic of a system for sequestrating CO₂ with negative carbon dioxide emissions. The system includes an incinerator 520, an 02 removal unit 530, a CO₂ conversion bioreactor 540, a CO conversion bioreactor 550, an organic acid conversion bioreactor 555, an acid recovery system 558, a H₂O electrolyzer 560, a CO₂ electrolyzer 570, a cell mass processing unit 580, and a renewable power source 590.

A biogenic material 510 is sent to the incinerator 520 to produce an incineration flue gas 522 and a steam 524. In general, the incineration flue gas 522 may contain 10% to 20% CO₂, 60% to 75% N₂, 10% to 15% H₂O and 3% to 5% O₂ by volume. The incineration flue gas 522 is then sent to the O₂ removal unit 530 to produce a fermentable CO₂-containing flue gas 532. The O₂ removal unit 530 could be a catalytic system assisted by the addition of methane or hydrogen depending on the availability of fuel that reduces O₂ concentration to 1 to 100 ppm. The removed O₂ 534 is recycled back to the incinerator to be used as an oxidant agent. An external CO₂-containing stream 512, which may be an industrial off-gas, is blended with the incineration flue gas 522 or the fermentable CO₂-containing flue gas 532 to increase CO₂ concentration. The fermentable CO₂-containing flue gas 532 is then provided to the CO₂ conversion bioreactor 540 with a CO₂ converting anaerobic bacteria. The CO₂ conversion bioreactor ferments the flue gas to produce one or more cell free organic acids containing permeate 541, 542, and 543 and an exhaust gas 548. In one embodiment, at least a portion of the CO₂ 536 from the fermentable CO₂-containing flue gas 532 is sent to the CO₂ electrolyzer 570. In another embodiment, the external CO₂-containing stream 512 is also supplied to the CO₂ electrolyzer 570. In still another embodiment, both a portion of the CO₂ 536 from the fermentable CO₂-containing flue gas 532 and the external CO₂-containing stream are provided to the CO₂ electrolyzer 570. The CO₂ electrolyzer 570 electrolysis the CO₂ it received into a CO stream 572 and an O₂ stream 574. The O₂ stream 574 is then used as an oxidant agent of the incinerator 520. Additional H₂ may be blended with the fermentable CO₂-containing flue gas 532 to form a desired H₂:CO₂ ratio for the CO₂ bioconversion. A H₂O electrolyzer 560 is configured to receive a water stream 514 and produce a H₂ stream 562 and an O₂ stream 564. The H₂ stream 562 may be used as a source of the additional H₂ or be directly added to the CO₂ conversion bioreactor 540.

The CO stream 572 produced by the CO₂ electrolyzer 570 and the cell free organic acids containing permeate 541 are sent to the CO conversion bioreactor 550 with a CO converting anaerobic bacteria. The CO converting anaerobic bacteria is capable of converting CO and organic acids into one or more oxygenated hydrocarbonaceous compounds 553. The vent gas 552 from the CO conversion bioreactor 550 contains CO₂ and can be recycled back to the CO₂ conversion bioreactor 540. A portion of the CO₂ 551 from the vent gas 552 may be also provided to the CO₂ electrolyzer 570. The cell free organic acids containing permeate 543 is sent to the organic acid conversion bioreactor 555 with a microorganism that is capable of converting organic acid into oxygenated hydrocarbonaceous compound 556. In one embodiment, the microorganism is a yeast and the oxygenated hydrocarbonaceous compound is a lipid. In another embodiment, the microorganism is Clostridium Kluyveri and the oxygenated hydrocarbonaceous compounds are medium chain carboxylic acids (MCCAs), such as hexanoic and octanoic acids. An acid recovery system 558 is configured to receive the cell free organic acid-containing permeate 542 to produce a concentrated organic acid 559 as a final product. In one aspect, the acid recovery system 558 is a reverse osmosis or a nanofiltration system. In one aspect, the acid recovery system 558 is a charged membrane system to concentrate organic acid through adsorption or rejection. In another aspect, the acid recovery system 558 is a solvent based azeotropic distillation system. In this aspect, the cell free organic acids containing permeate 543 is mixed with a solvent to extract the organic acid and then the solvent is separated out by an azeotropic distillation column to produce the concentrated organic acid 559.

Both the CO₂ conversion bioreactor 540, the CO conversion bioreactor 550, and the organic acid conversion bioreactor 555 continuously purge out cell mass during operation. Multiple cell separators and/or holding tanks are configured to concentrate the cell mass and produce cell mass containing suspension. The cell mass containing suspension 554 from the CO conversion bioreactor 550, the cell mass containing suspension 546 from the CO₂ conversion bioreactor 540, and the cell mass containing suspension 557 from the organic acid conversion bioreactor 555 are sent to the cell mass processing unit 580 to be processed into single cell protein 582.

Additional oxidant agents, such as air, steam and CO₂, may also be added to the incinerator 520. The heat steam 524 generated from the incinerator 520 is provided to the renewable power source 5900. The renewable power source produces renewable electricity 592 and 594, which are used as power source for the CO₂ electrolyzer 570 and the H₂ electrolyzer 560.

### EXAMPLES

### Example 1 Syngas Production by Gasification

A two-stage gasifier with a lower chamber and an upper chamber is used in this example. Carbonaceous material feedstock of dried wood chips with calorific value between 14 to 16 MJ/kg is introduced into the lower chamber. Oxygen is supplied to the gasifier at a rate of 350 to 450 kg per metric ton of the dried wood chips. The gaseous product from the lower chamber moves to the upper chamber. Ash is removed through the lower chamber. A raw syngas containing CO, H₂, CO₂, N₂ and other constituents (e.g. PM, tars, metals) is produced and removed from the upper chamber.

The raw syngas is then subjected to cleanup through a water spray column and an acid gas (e.g. HCl, H₂S) removal scrubber directly after the gasifier. A cyclone separator is used to remove PM and droplets. Table 1 shows the composition of the syngas produced from dried wood chips. Moisture/H₂O may be removed through cooler.

TABLE 1

| Component | mol. % |
|---|---|
| CO | 37.1% |
| H₂ | 34.6% |
| CO₂ | 16.9% |
| CH₄ | 0% |
| H₂O | 1.2% |
| N₂ - Others | 10.2% |
| Total | 100% |

The carbonaceous material feed is then changed to corn stover with 30% moisture content and calorific value between 11 to 13 MJ/kg. Oxygen is supplied to the gasifier at a rate of 225 to 325 kg per metric ton of the corn stover. Table 2 shows the composition of the syngas produced. Moisture/H₂O may be removed through cooler.

TABLE 2

| Component | mol. % |
|---|---|
| CO | 25% |
| H₂ | 34% |
| CO₂ | 29% |
| CH₄ | 0.1% |
| H₂O | 1% |
| N₂ - Others | 10.9% |
| Total | 100% |

### Example 2 Fermentation of Syngas Produced from Dried Wood Chips

A fermentation system with a CO conversion bioreactor and a CO₂ conversion bioreactor as shown in FIG. 2 is used to ferment the syngas produced from dried wood chips. Each bioreactor contains a liquid medium containing vitamins, trace metals, and salts as described herein. The CO converting bioreactor is started with actively growing Clostridium ljungdahlii at pH 4.6 and the CO₂ converting bioreactor is started with actively growing Acetobacterium woodii at pH 6.5.

The CO conversion bioreactor receives 4,004 m³ syngas produced from 2.8 metric ton of dried wood chips as described in Example 1 and produces 25 to 40 g/L ethanol while maintaining a CO conversion rate of approximately 90% and a specific ethanol productivity of 10-16 grams/day/gram of cells. The vent gas from the CO conversion bioreactor has 2% CO, 35% H₂, 50% CO₂, 0% CH₄ and 13% N₂ and other inert and is delivered to the CO₂ conversion bioreactor as a feed gas. An additional of 750 m³ H₂ gas is supplemented to the CO₂ conversion bioreactor. The CO₂ conversion bioreactor produces 10-25 g/L acetic acid with a CO₂ conversion rate of 95% to 97% and a specific acetic acid productivity of 10-15 grams/day/gram of cells. Acetic acid from the CO₂ conversion bioreactor is provided to the CO conversion bioreactor and is subsequently converted into ethanol by the Clostridium ljungdahlii bacteria. The exhaust gas from CO₂ conversion bioreactor contains 0% CO, 51% H₂, 7% CO₂, 0% CH₄ and 42% N₂ and other inert. The two-bioreactor fermentation system achieves 100% CO conversion, 95 to 97% CO₂ conversion, and 97.5 to 98.5% total carbon conversion.

About 95% of the CO₂ from the exhaust gas is separated out through a MDEA scrubber and sent to a CO₂ electrolyzer. CO generated from the CO₂ electrolyzer is sent to the CO conversion bioreactor to increase ethanol production. By utilizing the CO₂ electrolyzer, ethanol production is 373 kg per metric ton of the dried wood chips fed to the gasifier compared to about 357 kg per metric ton of dried wood chips fed to the gasifier without CO₂ electrolyzer. The rest of the CO₂ from the exhaust gas is removed by a scrubber to generate a carbon-free exhaust gas with 0% CO, 55% H₂, 0% CO₂, 0% CH₄ and 45% N₂ and other inert.

### Ethanol Production Per Ton Dried Wood Chips

Gasifier+CO conversion bioreactor: 210 to 250 kg/T

Gasifier+CO conversion bioreactor+CO₂ conversion bioreactor: 345 to 360 kg/T

Gasifier+CO₂ electrolyzer+CO conversion bioreactor+CO₂ conversion bioreactor: 365 to 380 kg/T

### Example 3 Fermentation of Syngas Produced from Corn Stover with 30% Moisture Content

A fermentation system with a CO conversion bioreactor and a CO₂ conversion bioreactor as shown in FIG. 2 is used to ferment the syngas produced from corn stover with 30% moisture content. Each bioreactor contains a liquid medium containing vitamins, trace metals, and salts as described herein. The CO converting bioreactor is started with actively growing Clostridium ljungdahlii at pH 4.6 and the CO₂ converting bioreactor is started with actively growing Acetobacterium woodii at pH 6.5.

6,044 m³ syngas is produced by gasifying 5.8 metric tons of corn stover with 30% moisture content as described in Example 1. 92% (1,600 m³) of the CO₂ in the syngas is separated out from the syngas through a MDEA scrubber. The CO-concentrated syngas after CO₂ separation contains 34.7% CO, 46.8% H₂, 3.4% CO₂, 0% CH₄, and 15.1% N₂ and other inert.

The CO conversion bioreactor receives the CO-concentrated syngas and produces 25-35 g/L ethanol while maintaining a CO conversion rate of approximately 90% and a specific ethanol productivity of 10-15 gram/day/gram of cells. The vent gas from the CO conversion bioreactor has 2.5% CO, 45% H₂, 31.3% CO₂, 0% CH₄, 21.2% N² and other inert and is delivered to the CO₂ conversion bioreactor as a feed gas.

The CO₂ conversion bioreactor receives the vent gas from the CO conversion bioreactor, the separated-out CO₂ stream and a supplemented H₂ stream of 3200 m³. The CO₂ bioconversion process produces 12 to 25 g/L acetic acid with a CO₂ conversion rate of 95% to 97% and a specific acetic acid productivity of 10-15 grams/day/gram of cells. Acetic acid from the CO₂ conversion bioreactor is provided to the CO conversion bioreactor and is subsequently converted into ethanol by the Clostridium ljungdahlii bacteria. Total ethanol produced is 2.1 metric tons. The exhaust gas from CO₂ conversion bioreactor contains 0% CO, 43.9% H₂, 4.1% CO₂, 0% CH₄, 52% N² and other inert. The two-bioreactor fermentation system achieves 100% CO conversion, 95 to 97% CO₂ conversion, and 97.5 to 98.5% total carbon conversion.

Alternatively, the 1600 m³ CO₂ stream may be sent to a CO₂ electrolyzer to produce a CO stream and an O₂ stream. The CO stream can be sent to the CO conversion bioreactor directly and the O₂ stream can be used as a fuel in the gasifier. 1600 m³ CO can be produced by consuming 9,440 kWh electricity. 1.05 metric tons of ethanol can be further produced by fermenting the 1600 m³ CO by the CO conversion bioreactor.

Ethanol Production Per Ton Corn Stover with 30% Moisture Carbonaceous Materials

Gasifier+CO conversion bioreactor: 130 to 200 kg/T

Gasifier+CO conversion bioreactor+CO₂ conversion bioreactor: 230 to 290 kg/T

Gasifier+CO₂ separation+CO conversion bioreactor+CO₂ conversion bioreactor: 240 to 310 kg/T

Gasifier+CO₂ separation+CO₂ electrolyzer+CO conversion bioreactor+CO₂ conversion bioreactor (calculated): 305 to 330 kg/T

It has been shown that (1) electrolyzing the CO₂ stream in a CO₂ electrolyzer and subsequent fermenting the electrolyzer generated CO in the CO conversion bioreactor provide more ethanol production per metric ton of carbonaceous materials than (2) directly fermenting the separated-out CO₂ stream in the CO₂ conversion bioreactor. Compared to approach (2), approach (1) consumes more electricity energy, needs less H₂, and produces O₂ for the gasifier. Both approaches may be taken to eliminate the CO₂ in the syngas produced from gasifying carbonaceous materials with low calorific value. Selection could be made by comparing the cost of H₂ consumption and electricity consumption.

While the disclosure herein disclosed has been described by means of specific embodiments, examples, and applications thereof, other and further variations could be devised without departing from the basic scope of the disclosure set forth in the claims that follow.

What is claimed is:

1. A fermentation process, comprising:
providing a feedstock to a gasifier to generate a CO-containing syngas;
passing the CO-containing syngas to a bioconversion system with at least one CO conversion bioreactor and one CO₂ conversion bioreactor;
recovering an alcohol from the bioconversion system;
passing a CO₂-containing stream from the bioconversion system to a CO₂ electrolyzer to produce a first O₂ stream and a CO stream; and
providing the first O₂ stream to the gasifier and the CO stream to the bioconversion system.

2. The process of claim 1 further comprising:
obtaining from the bioconversion system a fermentation liquid broth;
separating the fermentation liquid broth into a cell free permeate and a cell-containing suspension;
producing a single cell protein from the cell-containing suspension.

3. The process of claim 1 further comprising:
fermenting the CO-containing syngas in the at least one CO conversion bioreactor with a CO converting anaerobic bacteria to produce a cell free alcohol-containing permeate and a vent gas;
fermenting at least a portion of the vent gas in the at least one CO₂ conversion bioreactor with a CO₂ converting anaerobic bacteria to produce a cell free organic acid-containing permeate and an exhaust gas;
recycling the cell free organic acid-containing permeate to the at least one CO conversion bioreactor.

4. The process of claim 3 wherein the CO converting anaerobic bacteria is selected from the group consisting of Clostridium ljungdahlii, Clostridium autoethanogenum, Clostridium ragsdalei and Clostridium coskatii.

5. The process of claim 3 wherein the CO₂ converting anaerobic bacteria is select4ed from the group consisting of Acetogenium kivui, Acetoanaerobium noterae, Acetobacterium woodii, Alkalibaculum bacchi CP11 (ATCC BAA-1772), Moorella thermoacetica, Moorella thermoautotrophica, Ruminococcus productus, and Acetogenium kivui.

6. The process of claim 1 wherein the alcohol recovered is selected from the group consisting of ethanol and butanol.

7. The process of claim 1 wherein the process produces 300 kg or more alcohol per metric dry ton feedstock gasified.

8. The process of claim 1 wherein at least 95% of the carbon from carbon monoxide and carbon dioxide provided from the CO-containing syngas has been converted into the alcohol and the single cell protein.

9. The process of claim 1 wherein the process provides 99% or more CO conversion rate and 95% or more CO₂ conversion rate.

10. The process of claim 1 further comprising:
providing at least a portion of a second O₂ stream to the gasifier and at least a portion of a H₂ stream to the bioconversion system, wherein the second O₂ stream and the H₂ stream are produced by a water electrolyzer.

11. The process of claim 10 further comprising:
powering the CO₂ electrolyzer and the water electrolyzer with renewable electricity generated by a renewable power source.

12. The process of claim 10 further comprising:
recovering heat from the gasifier to produce a steam;
providing the steam to the renewable power source.

13. The process of claim 1, wherein the exhaust gas contains 10% or less CO₂ and 1% or less CO.

14. The process of claim 1 further comprising:
passing an additional oxidant to the gasifier.

15. A fermentation system, comprising:
a gasifier gasifying a feedstock to generate a CO-containing syngas;
a bioconversion system with at least one CO conversion bioreactor and one CO₂ conversion bioreactor receiving the CO-containing syngas and producing an alcohol and a CO₂-containing exhaust gas stream;
a CO₂ electrolyzer receiving the CO₂-containing exhaust gas stream to produce a first O₂ stream and a CO stream;
a pipeline recycling the CO stream to the bioconversion system; and
a pipeline recycling the first O₂ stream to the gasifier.

16. A fermentation process, comprising:
providing a feedstock to a gasifier to generate a CO-containing syngas with 20% or more CO₂ and 25% or less CO;
separating the CO-containing syngas into a CO₂ stream and a CO-concentrated fermentable syngas;
passing the CO-concentrated fermentable syngas to a CO conversion bioreactor to produce a cell free alcohol-containing permeate and a CO₂-containing vent gas;
recovering alcohol from the cell free alcohol-containing permeate;
passing the CO₂-containing vent gas and at least a portion of the CO₂ stream to a CO₂ conversion bioreactor to produce a cell free organic acid-containing permeate and a CO₂-containing exhaust gas; and
delivering the acid-containing permeate to the CO conversion bioreactor.

17. The process of claim 16 further comprising:
passing at least a portion of the CO₂-containing exhaust gas to a CO₂ electrolyzer to produce a first O₂ stream and a CO stream; and
providing the first O₂ stream to the gasifier and the CO stream to the CO conversion bioreactor.

18. The process of claim 16 further comprising:
passing at least a portion of the CO₂ stream to the CO₂ electrolyzer.

19. The process of claim 16 further comprising:
passing at least a portion of the CO₂ stream to the CO₂ conversion bioreactor.

20. A fermentation process, comprising:
providing a CO-containing gaseous substrate to a CO conversion bioreactor with a CO converting anaerobic bacteria to produce a cell free alcohol-containing permeate and a vent gas;
recovering the alcohol from the cell free alcohol-containing permeate;
passing at least a portion of the vent gas to a CO₂ electrolyzer to produce a CO stream;
providing the CO stream to the CO conversion bioreactor;
fermenting at least a portion of the vent gas in a CO₂ conversion bioreactor with a CO₂ converting anaerobic bacteria to produce a cell free organic acid-containing permeate and an exhaust gas; and
recycling at least a portion of the cell free organic acid-containing permeate to the CO conversion bioreactor, wherein the CO converting anaerobic bacteria converts at least a portion of the organic acid into the alcohol.

21. A fermentation system, comprising:
a CO conversion bioreactor fermenting an organic acid and a CO-containing gaseous substrate with a CO converting anaerobic bacteria to produce a cell free alcohol-containing permeate and a vent gas;
a CO₂ conversion bioreactor fermenting at least a portion of the vent gas with a CO₂ converting anaerobic bacteria to produce a cell free organic acid-containing permeate and an exhaust gas;
a pipeline recycling at least a portion of the cell free organic acid-containing permeate to the CO conversion bioreactor;
a distillation column recovering the alcohol from the cell free alcohol-containing permeate;
a CO₂ electrolyzer electrolyzing at least a portion of the vent gas and/or the exhaust gas to generate a CO stream; and
a pipeline recycling the CO stream to the CO conversion bioreactor.

22. A fermentation process, comprising:
providing a biogenic material to an incinerator to produce a CO₂-containing incineration flue gas;
fermenting at least a portion of the CO₂-containing incineration flue gas in a CO₂ conversion bioreactor with a CO₂ converting anaerobic bacteria to produce a cell free organic acid-containing permeate and an exhaust gas;
delivering a CO₂ stream to a CO₂ electrolyzer to produce a first O₂ stream and a CO stream;
passing the first O₂ stream to the incinerator;
fermenting the CO stream and at least a portion of the organic acid-containing permeate in a CO conversion bioreactor with a CO converting anaerobic bacteria to produce a cell free alcohol-containing permeate and a vent gas CO₂ stream;
delivering at least a portion of the vent gas CO₂ stream to the CO₂ conversion bioreactor.

23. The process of claim 22, further comprising:
removing O₂ from the CO₂-containing incineration flue gas;
sending the removed O₂ to the incinerator.

24. The process of claim 22, further comprising:
the CO₂ stream comprises CO₂ from the CO₂-containing incineration flue gas.

25. The process of claim 22, further comprising:
the CO₂ stream comprises CO₂ from an external industrial off-gas.

26. The process of claim 22, further comprising:
the CO₂ stream comprises CO₂ from both the CO₂-containing incineration flue gas and the external industrial off-gas.

27. The process of claim 22, further comprising:
providing at least a portion of a second O₂ stream to the incinerator and at least a portion of a H₂ stream to the CO₂ conversion bioreactor, wherein the second O₂ stream and the H₂ stream is produced by a water electrolyzer.

28. The process of claim 22 further comprising:
powering the CO₂ electrolyzer and the water electrolyzer with renewable electricity generated by a renewable power source.

29. The process of claim 22 further comprising:
recovering heat from the incinerator to produce a steam;
providing the steam to the renewable power source.

30. The process of claim 22 further comprising:
fermenting at least a portion of the organic acid-containing permeate with an organic acid converting microorganism to produce an oxygenated hydrocarbonaceous compound and a second vent gas CO₂ stream;
recycling the second vent gas CO₂ stream to the CO₂ conversion bioreactor.

31. The process of claim 30 wherein the organic acid converting microorganism is Clostridium Kluyveri and the oxygenated hydrocarbonaceous compound is MCCAs.

32. The process of claim 30 wherein the organic acid converting microorganism is a yeast selected from the group consisting of Cutaneotrichosporon and Oleaginosum, and the oxygenated hydrocarbonaceous compound is lipid.

33. The process of claim 22 further comprising:
recovering the organic acid from at least a portion of the organic acid-containing permeate through an acid recovery system.

34. A fermentation system, comprising:
an incinerator incinerating a biogenic material to produce a CO₂-containing incineration flue gas;
a CO₂ conversion bioreactor fermenting at least a portion of the CO₂-containing incineration flue gas to produce an organic acid-containing permeate and an exhaust gas;
a CO₂ electrolyzer receiving a CO₂ stream to produce a first O₂ stream and a CO stream;
a pipeline recycling the first O₂ stream to the incinerator;
a CO conversion bioreactor fermenting the CO stream and the organic acid-containing permeate with a CO converting anaerobic bacteria to produce a cell free alcohol-containing permeate and a vent gas CO₂ stream; and
a pipeline recycling at least a portion of the vent gas CO₂ stream to the CO₂ conversion bioreactor.
